(19) United States
(12) Patent Application Publication
(10) Pub. No.: US 2018/0360335 A1
(43) Pub. Date: Dec. 20, 2018

(54) METHOD AND SYSTEM FOR MODIFYING THE PROPERTIES OF A CONSUMABLE LIQUID

(71) Applicant: Facecontrol, Inc., Hallandale Beach, FL (US)

(72) Inventors: Oksana Zatvan, Miami, FL (US); Alexander Zatvan, Moscow (RU); Konstantin Pronko, Moscow (RU); Nikita Pronko, Moscow (RU); Alexey Diashev, Moscow (RU)

(73) Assignee: Facecontrol, Inc., Hallandale Beach, FL (US)

(21) Appl. No.: 15/625,746

(22) Filed: Jun. 16, 2017

(51) Int. Cl. A61B 5/04; A61B 5/00; A61B 5/024

(52) U.S. Cl. CPC A61B 5/04012; A61B 5/0077; A61B 5/02405; A61B 5/0022; A61B 5/742; A61B 5/7278

(57) ABSTRACT

A computer-implemented method and system is disclosed for modifying the properties of a consumable liquid to preventively treat or functionally correct human health conditions. The system includes a database storing human health conditions and an electronic device having a processor communicatively coupled to the database. The electronic device captures at least one visual image or video of a face of a person over a defined period of time via a digital camera. The captured images or video are processed by the electronic device to obtain the person's cardiointervalogram. From the cardiointervalogram, the electronic device identifies a particular human health condition of the person by comparing slow wave parameters of the cardiointervalogram to the corresponding human health conditions stored in the database. The electronic device then determines specified properties for light waves and/or acoustic waves based on the identified health condition(s). Light waves and/or acoustic waves having the specified properties are thereafter emitted from a light source and/or audio source to the consumable liquid to modify the properties of the consumable liquid to preventively treat or functionally correct the health condition(s).

### FIELD OF THE INVENTION

The present invention relates generally to preventive treatment and functional correction of human health conditions, and, more particularly, a method and system for modifying the properties of a consumable liquid based on heart rate variability (HRV), to effectuate such preventive treatment.

### BACKGROUND OF THE INVENTION

A cardiointervalogram represents a person's HRV, which is reflective of an effect of the various regulatory systems on a heart rate (sympathetic and parasympathetic divisions of the autonomic nervous system and an effect of the humoral system). Current activity of sympathetic and parasympathetic divisions is a result of multiloop and multistage reaction of the blood circulation system, which changes its parameters in time in order to achieve optimal adaptive response reflecting the adaptive reaction of the integral organism.

The sympathetic nervous system is responsible for the mobilization of domestic resources of the body, while the parasympathetic system is responsible for relaxation, recreation, preservation and accumulation of important energy. Humoral regulation is one of the earliest evolutionary mechanisms of vital processes regulation in the body, carried through the body fluids (e.g., blood, lymph, and tissue fluid) with the help of hormones excreted by cells, organs, and tissues. In humans, humoral regulation is subject to the neural regulation. The current activity of sympathetic and parasympathetic divisions is the result of the multiloop and multistage reaction of the blood circulation system changing its parameters in time in order to achieve optimal adaptive response that reflects the adaptive responses of the organism. Adaptive reactions are individual and implemented by different people with different degrees of participation in functional systems, which have feedback changing in time and having a variable functional organization. A healthy person's indicators of a cardiointervalogram in standard conditions of registration have confidential persistence and characterize vegetative homeostasis. Difficulties in describing the meaning of the terms of constant or particular time. For example, the regulation of a heart rate effects in 0.1-1.5 seconds. Influence on the water by electromagnetic and mechanical waves and its following consumption allows to regulate a human's homeostasis and compensate his or her energy expenditure, in addition to increasing the adaptive capacity of the human body.

The cardiointervalogram can be represented by four principal wave peaks, frequency and amplitude (power), which correspond to the principal waves ULF (ultra-low frequency), VLF (very low frequency—<0.04 Hz), LF (low frequency—0.04 to 0.15 Hz), and HF (high frequency—0.15 to 0.4 Hz). HF waves are associated with parasympathetic activity, while LF frequency waves are associated with sympathetic nervous system. Thus, these waves can be used to identify particular human health conditions.

It would therefore be desirable to provide a method and system that uses a person's cardiointervalogram to generates light waves and/or sound waves using the slow wave parameters of the cardiointervalogram to modify the properties of a consumable liquid to preventively treat and functionally correct human health conditions.

### SUMMARY OF THE INVENTION

The present invention provides a system and method for modifying the properties of a consumable liquid to preventively treat and functionally correct human health conditions.

With the foregoing and other objects in view, there is provided, in accordance with the invention, a computer-implemented method of modifying the properties of a consumable liquid to preventively treat and functionally correct human health conditions. The method includes executing computer program instructions stored in memory by at least one processor of an electronic device to receive data representing a person's heart rate variability (HRV) according to an cardiointervalogram, and identify from the cardiointervalogram a particular human health condition of the person by comparing slow wave parameters of the cardiointervalogram to characteristics of corresponding human health conditions stored in a database. The method further includes determining specified properties for at least one of light and acoustic waves to be applied to the consumable liquid to preventively treat and functionally correct the particular human health condition. Based on this determination, the light and/or acoustic waves having the specified properties are emitted from at least one of a light source and an audio source coupled to the processor of the electronic device, to the consumable liquid so as to modify properties of the consumable liquid to preventively treat and functionally correct the particular human health condition.

In accordance with another feature of the present invention, the method further includes capturing at least one visual image or video of a face of a person via a digital camera of the electronic device over a defined period of time. The captured image or video is processed to obtain the person's cardiointervalogram.

In accordance with yet another feature of the present invention, the method further includes rendering the cardiointervalogram of the person in real time on a display.

In accordance with yet another feature of the present invention, the method further includes rendering, a pulsogram of the slow wave parameters of the cardiointervalogram on a display.

In accordance with still another feature of the present invention, the method further includes capturing the at least one image or video of the person with a digital camera having a pixel resolution of at least 640×480 pixels.

In accordance with yet another feature of the present invention, the method further includes prompting the person to be physically disposed in front of a camera lens of the digital camera within a single separation distance from the camera lens during the defined period of time.

In accordance with still another feature of the present invention, the method further includes using the slow wave parameters of the cardiointervalogram and properties of light for preventively treating and functionally correcting the corresponding health conditions stored in the database, to select at least one color and properties of the color including frequency, phase and intensity, for emission to the consumable liquid to modify the properties of the consumable liquid.

In accordance with yet another feature of the present invention, the method further includes using the slow wave parameters of the cardiointervalogram and properties of sound for preventively treating and functionally correcting the corresponding health conditions stored in the database, to select at least one sound and properties of the sound including frequency, phase and amplitude, for emission to the consumable liquid to modify the properties of the consumable liquid.

In accordance with still another feature of the present invention, the consumable liquid is water and, by emitting the at least one of the light and acoustic waves having the specified properties, at least one of electronic water saturation, biocatalytic water activity, thermodynamic changes, water memory, and water memory density distribution by energetic levels of the consumable liquid is modified.

In accordance with another aspect of the present invention, there is provided a system for modifying the properties of a consumable liquid to preventively treat and functionally correct human health conditions. The system includes a database storing human health conditions, and an electronic device communicatively coupled to the database and including at least one processor, a non-transitory memory, a display, an audio source, a light source, and a digital camera communicatively coupled to one another. The electronic device further includes a set of computer program instructions stored in the non-transitory memory and executable by the at least one processor. The set of computer instructions may, in accordance with another feature of the present invention, include instructions for receiving data representing a cardiointervalogram of a person; identifying from the cardiointervalogram of the person, a particular human health condition of the person by comparing slow wave parameters of the cardiointervalogram to characteristics of the corresponding human health conditions stored in the database; determining specified properties for at least one of light and acoustic waves to be applied to the consumable liquid to preventively treat and functionally correct the particular human health condition; and emitting at least one of light and acoustic waves having the specified properties, from at least one of the light source and the audio source to the consumable liquid to modify properties of the consumable liquid to preventively treat and functionally correct the particular human health condition.

In accordance with another feature of the present invention, the set of computer instructions includes instructions for causing the digital camera to capture at least one visual image or video of a face of a person over a defined period of time. The captured at least one visual image or video is then processed to obtain the person's cardiointervalogram.

In accordance with yet another feature of the present invention, the set of computer instructions includes instructions for rendering the cardiointervalogram of the person in real time on a display.

In accordance with yet another feature of the present invention, the set of computer instructions includes instructions for rendering a pulsogram of the slow wave parameters of the cardiointervalogram on a display.

In accordance with still another feature of the present invention, the digital camera has a pixel resolution of at least 640×480 pixels.

In accordance with yet another feature of the present invention, the set of program instructions includes instructions for prompting the person to be physically disposed in front of a camera lens of the digital camera within a single separation distance from the camera lens during the defined period of time.

In accordance with still another feature of the present invention, the set of program instructions includes instructions for using the slow wave parameters of the cardiointervalogram and properties of light for preventively treating and functionally correcting the corresponding health conditions stored in the database, to select at least one color and properties of the color including frequency, phase and intensity, for emission to the consumable liquid to modify the properties of the consumable liquid.

In accordance with yet another feature of the present invention, the set of program instructions further program instructions for using the slow wave parameters of the cardiointervalogram and properties of sound for preventively treating and functionally correcting the corresponding health conditions stored in the database, to select at least one sound and properties of the sound including frequency, phase and amplitude, for emission to the consumable liquid to modify the properties of the consumable liquid.

In accordance with yet another feature of the present invention, there is provided a peripheral device communicatively coupled to the electronic device by either a wired or wireless connection. The peripheral device is configured with at least one light source and at least one audio source for emitting at least one of the light and the acoustic waves having the specified properties to a container storing the consumable liquid. The peripheral device can be constructed and arranged as a waterproof unit, to enable it to be directly immersed in the consumable liquid.

Although the invention is illustrated and described herein as embodied in a system and method for modifying the properties of a consumable liquid to preventively treat and functionally correct human health conditions, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time.

As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. In this document, the term “longitudinal” should be understood to mean in a direction corresponding to an elongated direction of a personal computing device from one terminating end to an opposing terminating end. The terms “program,” “software application,” and the like as used herein, are defined as a sequence of instructions designed for execution on a computer system. A “program,” “computer program,” or “software application” may include a subroutine, a function, a procedure, an object method, an object implementation, an executable application, an applet, a servlet, a source code, an object code, a shared library/dynamic load library and/or other sequence of instructions designed for execution on a computer system.

### BRIEF DESCRIPTION OF THE DRAWINGS

The accompanying figures, where like reference numerals refer to identical or functionally similar elements throughout the separate views and which together with the detailed description below and references to prior-art methods and devices are incorporated in and form part of the specification, serve to further illustrate various embodiments and explain various principles and advantages all in accordance with the present invention.

FIG. 1 is a block diagram of an exemplary distributed data processing network with a personal mobile computing device, a personal computer (PC), and a server/database in accordance with an embodiment of the present invention;

FIG. 2 is a block diagram of an exemplary electronic device, such as a personal mobile computing device, in accordance with the present invention;

FIG. 3 is a process flow chart representing an exemplary method of modifying the properties of a consumable liquid to preventively treat and functionally correct human health conditions based on a person's cardiointervalogram in accordance with an embodiment of the present invention;

FIG. 4 is a schematic of a user positioned relative to a personal computing device in accordance with an aspect of the present invention;

FIG. 5 is a screenshot depicting an exemplary user's cardiointervalogram obtained in accordance with an aspect of the present invention;

FIG. 6 is a screenshot of a final (post-wave) signal, after processing the pulsogram by modulation with waves of previously specified parameters, and the calculated color spectrum in accordance with an aspect of the present invention;

FIG. 7 is a screenshot of another final (post-wave) signal corresponding to an exemplary human health condition in accordance with an aspect of the present invention;

FIG. 8A is an illustration of a first exemplary embodiment in which headphones are coupled to a transparent container containing a consumable liquid for modifying the properties of the consumable liquid in accordance with an aspect of the present invention;

FIG. 8B is a depiction of another embodiment in which the transparent container containing a consumable liquid is placed above the display of a personal computing device for modifying the properties of the consumable liquid by emitting light waves having specified properties from the display;

FIG. 8C is an illustration of an embodiment similar to FIG. 8B, where the personal computing device is placed above the container;

FIG. 8D depicts one embodiment of a peripheral device that communicates with the personal computing device, and which is configured to receive a transparent container for modification of the consumable liquid in accordance with an aspect of the present invention;

FIG. 8E is a fragmentary cross-sectional view of the peripheral device of FIG. 8D;

FIG. 8F illustrates a first embodiment of a waterproof peripheral device that may be immersed in the consumable liquid to effectuate modification thereof in accordance with an aspect of the present invention;

FIG. 8G shows the waterproof peripheral device of FIG. 8F immersed in the consumable liquid;

FIG. 8H illustrates another embodiment of a waterproof peripheral device configured as a tablet that may be immersed in a consumable liquid;

FIG. 8I is another view of the waterproof peripheral device of FIG. 8H;

FIG. 8J is an illustration of the peripheral device of FIGS. 8H and 8I, immersed in the consumable liquid to effectuate modification thereof in accordance with an aspect of the present invention;

FIG. 9 pictorially illustrates a pulsogram (HRV) obtained for a test subject in accordance with an aspect of the present invention;

FIG. 10 depicts the initially specified characteristics of the main parameters of an acoustic wave in accordance with an aspect of the present invention;

FIG. 11 illustrates the post wave parameters obtained from post transformation of the pulsogram of FIG. 9;

FIG. 12 depicts the final (post-wave) signal reflecting a patient's stressed state, which is the signal (sound and color) for playback, in accordance with an aspect of the present invention; and

FIG. 13 is a block diagram of a plurality of logical structures performing various steps of the inventive process in accordance with an embodiment of the present invention.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

The present invention provides a novel and efficient computer-implemented method and system for modifying the properties of a consumable liquid to preventively treat and functionally correct human health conditions based on a person's heart rate variability (HRV) represented by a cardiointervalogram.

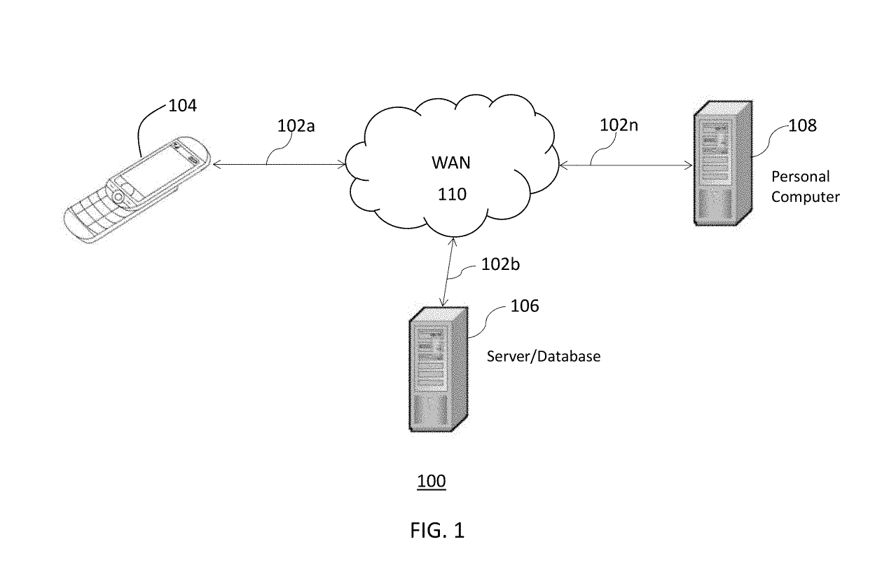

Referring now to FIG. 1, one embodiment of the present invention is shown as a block diagram, illustrating an exemplary network of data processing system in which the present invention may be implemented. FIG. 1 shows several advantageous features of the present invention, but, as will be described below, the invention can be provided in several shapes, sizes, combinations of features and components, and varying numbers and functions of the components. The first example of a network 100, as shown in FIG. 1, includes connections 102a-n, which are the medium used to provide communication links between various devices and computers connected together within the network 100. The connections 102a-n may be wired or wireless connections. A few exemplary wired connections are cable, phone line, and fiber optic. Exemplary wireless connections include radio frequency (RF) and infrared radiation (IR) transmission. Many other wired and wireless connections are known in the art and can be used with the present invention.

In the depicted example, the network 100 includes an electronic device such as a personal mobile computing device 104, a server 106, and a personal computer 108. The personal mobile computing device 104 can be operable to execute programming instructions embodied in a software application that can be received from the server 106 via a wide area network (WAN) 110. In other embodiments, the personal computer 108 is operable to execute the programming instructions received from the server 106 over the WAN 110. In yet other embodiments, the software application is a web-based software application, a desktop software application, or a mobile device software app. In one embodiment, the WAN is the Internet. The Internet represents a worldwide collection of networks and gateways that use the TCP/IP suite of protocols to communicate with one another. At the heart of the Internet is a backbone of high-speed data communication lines between major nodes or host computers, consisting of thousands of commercial, government, educational and other computer systems that route data and messages. Of course, the network 100 also may be implemented as a number of different types of networks, such as for example, an Intranet, a local area network (LAN), or a cellular network. FIG. 1 is intended as an example, and not as an architectural limitation for the present invention.

The server 106 can be seen as a computer that manages access to a centralized resource or database. In some embodiments, users of personal mobile computing device 104 can request the software application embodying an exemplary method of the present invention. The server 106 can receive, process, and satisfy the request by sending the software application to the personal mobile computing device 104 via the WAN 110. In yet other embodiments, the personal computer 108 can request the software application and the server 106 can receive, process, and satisfy the request by sending the software application to the personal computer 108 via the WAN 110.

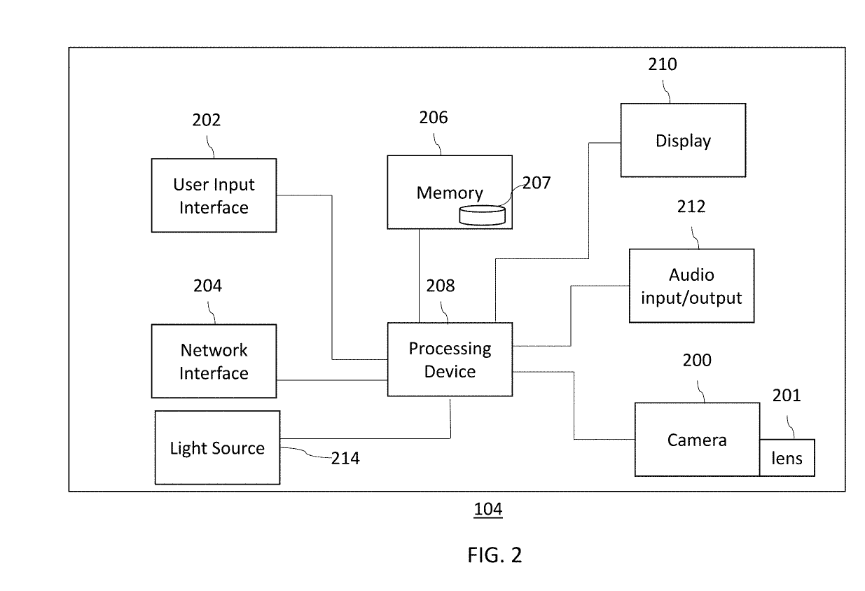

With reference now to FIG. 2, the personal computing device 104 is illustrated in a block diagram. The personal computing device 104 includes a camera 200, a user input interface 202, a network interface 204, a memory 206, a processing device 208, a computer display 210, an audio input/output 212, and a light source 214. The light source 214 can be a source of white light, or any other color of the spectrum, or a flashlight. Moreover, the light source 214 can be a separate device that is networked to the personal mobile computing device 104.

The camera 200 may include a camera lens 201 and may be operable to capture still images, as well as, video. The camera 200 is preferably a digital camera so that the images may be stored in the memory 206 and processed by the processing device 208. The camera 200 may be communicatively coupled to a microphone for capturing audio, as well as, simultaneous visual video images. The camera 200 is preferably operable to capture images having a pixel resolution of at least 640×480 pixels in order to provide a high-resolution image for interpreting and analyzing the images in accordance with techniques described herein, and generally known in the art. Cameras having a lesser quality may not be operable to provide high resolution images that may be required to accurately determine a user-food preference from captured images within a reasonable degree of error.

The user input interface 202 functions to provide the user a method of providing input to the personal computing device 104. The user input interface 202 may also facilitate interaction between the user and the device 104. The user input interface 202 may be a keypad providing a variety of user input operations. For example, the keypad may include alphanumeric keys for allowing entry of alphanumeric information (e.g. telephone numbers, contact information, text, etc.). The user input interface 202 may include special function keys (e.g. a camera shutter button, volume control buttons, back buttons, home button, etc.), navigation and select keys, a pointing device, and the like. Keys, buttons, and/or keypads may be implemented as a touchscreen associated with the computer display 210 of the type known in the art. The touchscreen may also provide output or feedback to the user, such as haptic feedback or orientation adjustments of the keypad according to sensor signals received by motion detectors, such as an accelerometer, located within the device 104.

The network interfaces 204 may include one or more network interface cards (NIC) or a network controller. In some embodiments, the network interface 204 may include a personal area network (PAN) interface. The PAN interface may provide the capability for the personal computing device 104 to network using a short-range communication protocol, for example, a Bluetooth communication protocol. The PAN interface may permit one personal computing device 104 to connect wirelessly to another personal computing device 104 via a peer-to-peer connection.

The network interfaces 204 may also include a local area network (LAN) interface. The LAN interface may be, for example, an interface to a wireless LAN, such as a Wi-Fi network. The range of the LAN interface may generally exceed the range available via the PAN interface. Typically, a connection between two electronic devices via the LAN interface may involve communication through a network router or other intermediary device.

Additionally, the network interfaces 204 may include the capability to connect to a wide area network (WAN) via a WAN interface. The WAN interface may permit a connection to, for example, a cellular mobile communications network. The WAN interface may include communications circuitry, such as an antenna coupled to a radio circuit having a transceiver for transmitting and receiving radio signals via the antenna. The radio circuit may be configured to operate in a mobile communications network, including but not limited to global systems for mobile communications (GSM), code division multiple access (CDMA), wideband CDMA (WCDMA), and the like.

The personal computing device 104 may also include a near field communication (NFC) interface. The NFC interface may allow for extremely close-range communication at relatively low data rates (e.g., 424 kb/s). The NFC interface may take place via magnetic field induction, allowing the NFC interface to communicate with other NFC interfaces located on other mobile computing devices 104 or to retrieve information from tags having radio frequency identification (RFID) circuitry. The NFC interface may enable initiation and/or facilitation of data transfer from one personal computing device 104 to another computing device 104 with an extremely close range (e.g. 4 centimeters).

Memory 206 associated with the device 104 may be, for example, one or more buffer, a flash memory, or non-volatile memory, such as random access memory (RAM). The personal computing device 104 may also include non-volatile storage. The non-volatile storage may represent any suitable storage medium, such as a hard disk drive or non-volatile memory, such as flash memory. The memory 206 may include at least one database 207 to be described in more detail below, which is communicatively coupled to the processing device 208 of the personal mobile computing device 104. In an embodiment in which the database 207 is considered at least a portion of the memory 206 on the personal computing device 104, such communicative coupling may be a hard-wired conductive connection. In an embodiment in which the database 207 is considered a remote database 106 accessible over, for example, a long-distance network, such as the WAN 110, such communicative coupling may be via the network interface 204 on the personal mobile computing device 104. The term “database” is intended in a broad sense to mean an organized collection of data that is stored in a non-transitory-type memory and is accessible by a processing device for utilizing the collection of data to perform computer processing tasks.

The processing device 208 can be, for example, a central processing unit (CPU), a microcontroller, or a microprocessing device, including a “general purpose” microprocessing device or a special purpose microprocessing device. The processing device 208 executes code stored in memory 206 in order to carry out operation/instructions of the personal mobile computing device 104. The processing device 208 may provide the processing capability to execute an operating system, run various applications, and provide processing for one or more of the techniques described herein.

The computer display 210 displays information to the user such as an operating state, time, telephone numbers, various menus, application icons, pull-down menus, and the like. The computer display 210 may be used to present various images, text, graphics, or videos to the user, such as photographs, mobile television content, Internet webpages, and mobile application interfaces. More specifically, the display 210 may be configured to display the cardiointervalogram of the user as described below. The computer display 210 may be any type of suitable display, such as a liquid-crystal display (LCD), a plasma display, a light-emitting diode (LED) display, or the like. The computer display 210 may also function as a source of white light or any other color of the spectrum, or function as a flashlight.

The personal computing device 104 may include audio input and output structures 212, such as a microphone for receiving audio signals from a user and/or a speaker for outputting audio signals, such as audio recordings associated with the user's speech and/or any emitted sound(s), movements, and the like. The personal computing device 104 may also include an audio port for connection to peripheral audio input and output structures, such as a headset, or peripheral speakers or microphones. In accordance with an embodiment of the invention, acoustic signals are generated based on the user's cardiointervalogram, and emitted from the audio output to modify the properties of a consumable liquid as further described below. Similarly, light signals are generated based on the user's cardiointervalogram, and emitted from the light source 214 to modify the properties of the consumable liquid as further described below.

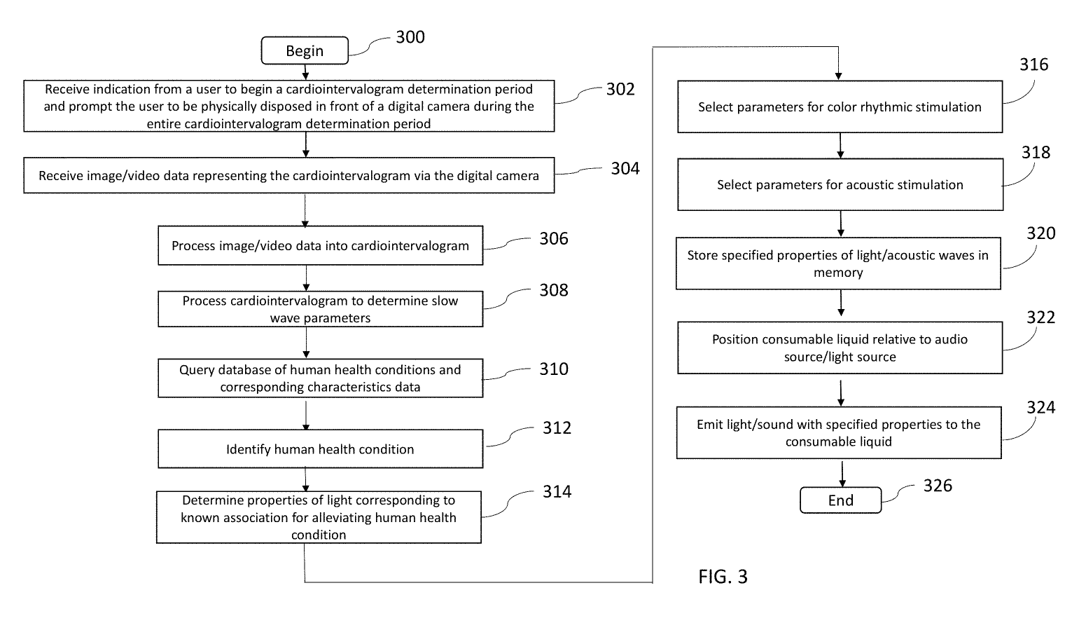

FIGS. 1-2 and 4-13 will be described in conjunction with the process flow chart of FIG. 3. Although FIG. 3 shows a specific order of executing the process steps, the order of executing the steps may be changed relative to the order shown in certain embodiments. Also, two or more blocks shown in succession may be executed concurrently or with partial concurrence in some embodiments. Certain steps may also be omitted in FIG. 3 for the sake of brevity. In some embodiments, some or all of the process steps included in FIG. 3 can be combined into a single process.

The exemplary process, depicted in FIG. 3, begins at step 300 and proceeds to step 302, where an indication is received from a user to initiate the cardiointervalogram determination. The indication from the user to begin is received via the user-input interface 202 on the personal computing device 104. The user may be provided with a “begin” or “start” button for the user to select to indicate that the user is ready to begin the process of determining the cardiointervalogram. The user maintains his or her orientation relative to the camera 200 for a defined period of time, typically at least approximately 30 seconds, or sufficiently enough for the cardiointervalogram to be generated, which is referred to as the “determination period.” It should be understood by persons of ordinary skill in the art that there are a number of other ways in which the user may input a “begin” or other initiation-type command, such as, an audio voice recognition command, or other methods and structures for inputting a user command into the personal computing device 104. In a further embodiment, after the user inputs a command to begin the defined cardiointervalogram determination period, the display 210 and/or audio output 212 may countdown or otherwise prepare the user to position himself/herself correctly prior to the determination period. In an exemplary embodiment, the display 210 counts down from 10 to give the user a set number of seconds, such as for example., 5 to 10 seconds, to position himself/herself correctly. It is important that the user physically position himself/herself correctly for the camera 200 to be able to capture images/video of the user during the determination period. Testing has shown that large facial obstructions, such as thick-rimmed glasses, will affect the cardiointervalogram reading of the user. Therefore, it is preferred that the user be free of such extraneous facial obstructions during the determination period.

During the determination period, the user should remain within a field of view of consistent distance the camera lens 201 of the camera 200. The user need not strictly fix his or her position in front of the camera 200. The user may move and turn his or her heard. If the user moves out of the field of vision sufficiently for the image/video to be lost, a signal such as sound and/or video, can be emitted by the personal computing device to indicate that the process has paused until the user returns his or her face to the camera's field of view to enable registering the cardiointervalogram. In one exemplary embodiment, the user may be prompted by the personal computing device 104 to be physically disposed in front of the camera lens 201 of the camera 200 within the field of view of the camera “FV” as illustrated in FIG. 4. The user may be prompted by the personal computing device 104 such as, for example, by a visual message 400 stating for example, “closer”, “further”, “remain fixed” or the like, which is displayed on the display 210 and/or an audio message output via the audio output 212 of the personal computing device 104 to ensure the user remains within the field of view suitable for the camera 200 to record the images/video for processing.

In step 304 the processor 208 of personal computing device 104 executes computer program instructions stored in a memory 206 to receive data the data representing the cardiointervalogram. In one embodiment, the user's expressions may be captured as a video, and/or one or more still digital images of the user. In a further embodiment, the face may be captured as at least two chronological still images captured within a specified time interval. In a particular embodiment, the camera 200 includes a video and/or one or more still digital images of the user's face. The captured images/video enable observation of blood filling the blood vessels in the face. In other embodiments, the camera 200 may also depict other physical body parts of the user from which biological/physiological characteristics can be identified and interpreted, and then processed to determine the user's cardiointervalogram. In other embodiments, the personal computing device 104 may include or be coupled to other sensors operable to capture the physiological/biological state of the user so as to determine the user's cardiointervalogram.

In step 306, the processor 208 executes computer program instructions stored in the memory 206 process the image/video data and convert this data into to the user's cardiointervalogram.

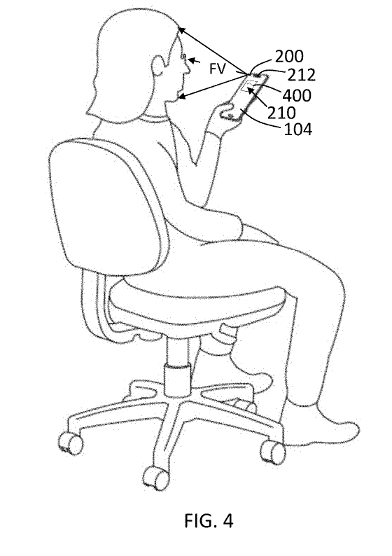

Referring to FIG. 5, there is shown a screenshot 500 depicting an exemplary user's cardiointervalogram 502 obtained from processing the captured visual images/video. In one embodiment, the cardiointervalogram 502 may be rendered in real time on the display 210 of the same electronic device that captures the images/video, or alternatively, the cardiointervalogram 402 may be rendered on the display of a separate, networked electronic device. For the purpose of illustration, the display of the computing device depicted in FIG. 5 is shown as a conventional desktop display.

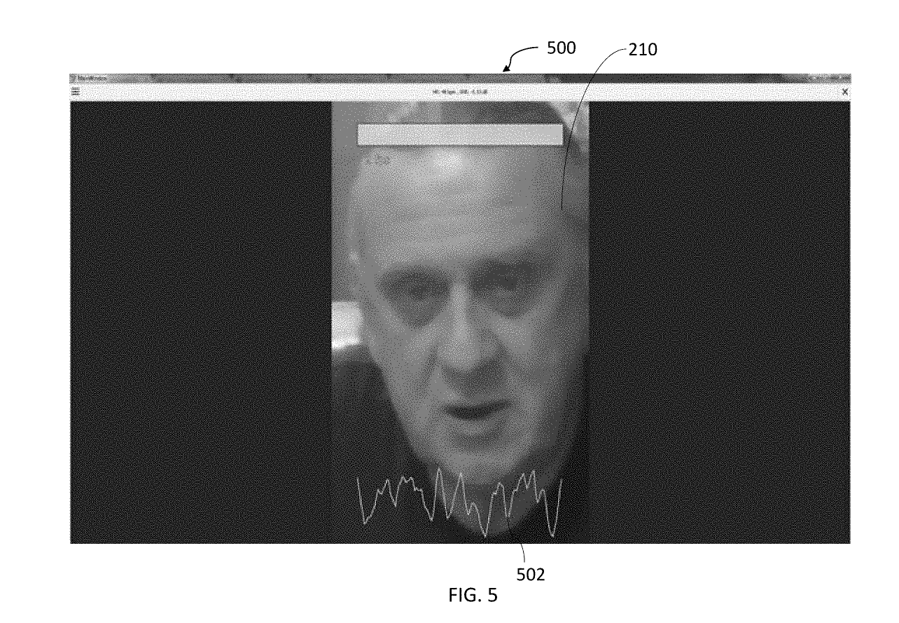

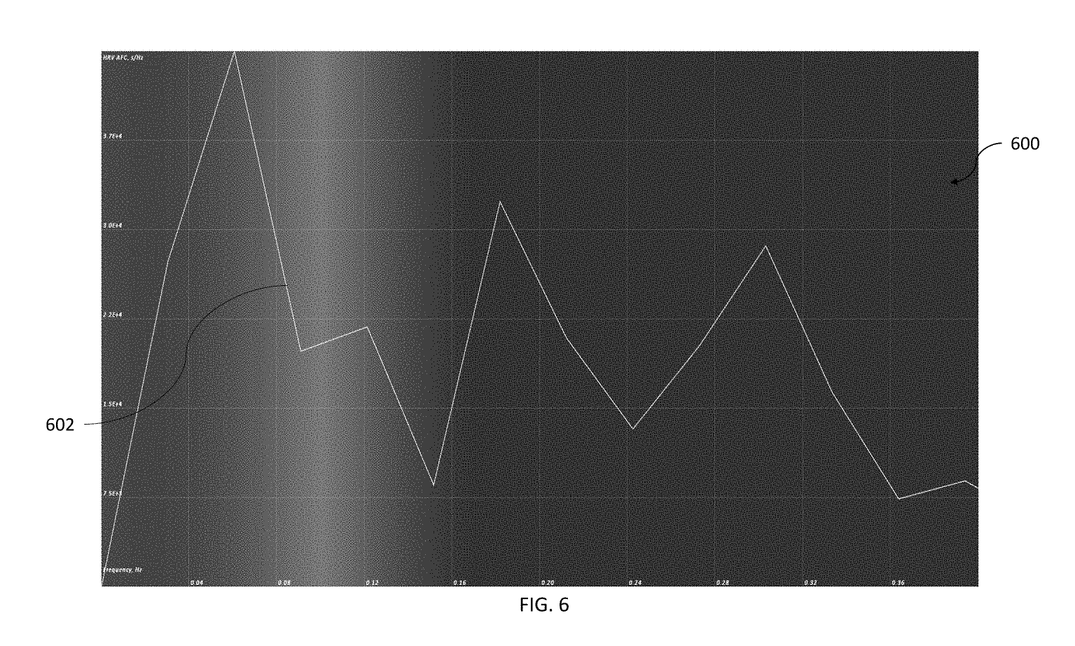

Referring again to FIG. 3, in step 308 processor 208 executes program instructions stored in memory 206 to process the user's cardiointervalogram to determine the slow wave parameters (cardiointervalogramms), consisting of four principle peaks, frequency and amplitude (power), which correspond to the principal waves ULF (ultra-low frequency), VLF (very low frequency—<0.04 Hz), LF (low frequency—0.04 to 0.15 Hz), and HF (high frequency—0.15 to 0.4 Hz). FIG. 6 is an exemplary screenshot 600 of a final (post-wave) signal 602, after processing the pulsogram by modulation with waves of previously specified parameters, and the calculated color spectrum displaying these slow wave parameters for a particular cardiointervalogram.

Referring again to FIG. 3, in step 310, the processor 208 queries the database 207, which stores a variety of human health conditions and corresponding characteristics data that may be matched to the slow wave parameters of cardiointervalogramms. The process then proceeds to step 312, where using the particular human health condition of the user is identified by comparing the slow wave parameters of the cardiointervalogram to the characteristics for human health conditions stored in the database 207.

In step 314, the processor 208 determines properties of light (frequency, phase, brightness, time of exposure and the like) and/or sound that correspond to known associations for alleviating certain human health conditions. In an exemplary expedient, the following table demonstrates the correlation between various colors and the effect on the human psychoemotional state. Certain colors associations can have a stimulating, activating or sometimes exciting effect, while others can have an opposite calming and suppressing effect. The predominant reference point is wave length (frequency), where the longer the wavelength, the stronger the stimulating effect. Accordingly, a red color has the greatest stimulating effect. Orange and yellow colors, i.e., warm colors, have a minimum stimulating effect. The shorter the wavelength, the weaker the sedative effect. A blue color, therefore, has a greatest sedating effect. The following table is illustrative:

| Color | Organs | Effects |
|---|---|---|
| Red | heart, arteries and vines | stimulating effect on blood-circulation organs, |
| | (blooms), liver, kidneys, | increase blood pressure; increases hemoglobin |
| | rectum | content in blood (prevents anemia); promotes |
| | | heat generation; controls liver and kidneys |
| | | activity; facilitates muscular relaxation; helps in |
| | | case of menstrual disorders and stimulates sexual |
| | | activity |
| Orange | spleen, pancreas, small | consolidating effect on body vitality; provides |
| | intestine, respiratory system | spleen and pancreas with energy; fortify |
| | | respiratory system |
| Yellow | nerves, brain, lymphatic | stimulates intellectual abilities; has cleansing |
| | system, gall bladder, | effect on digestive organs, liver, skin; provides |
| | stomach, duodenum | bile flow; restores mineral reserves content and |
| | | reduces body acidity |
| Green | nerves, muscles, bones, | calming effect; antiseptic, antimicrobic |
| | tendons, enzymes and | properties; strengthens muscles and tissues; |
| | hormones | stimulates hypophysis; has calming effect for |
| | | chronic diseases |
| Light-blue | nerves, larynx, epididymis, | eases pain, has significant effect and |
| | eyes, ears, nose | vasoconstrictive action; suppresses inflammatory |
| | | processes, has antiseptic action; antipyretic |
| | | action; good influence in case of nervous |
| | | breakdowns, fatigue, insomnia |
| Blue | respiratory system, nervous | beneficial effect on respiratory organs (very |
| | system, thyroid, tonsils, blind | effective in case of bronchitis, pneumonia and |
| | gut | asthma); controls thyroid activity; reduces |
| | | inflammatory processes in blind gut |
| | | (appendicitis) and tonsils; helps to stop bleedings, |
| | | fast cicatrization and wound healing; analgesic |
| | | action for various types of pain |
| Violet | nerves, glands, first of all | reduces temperature, decreases pain; good |
| | hypophysis, lymphatic | influence in case of hard working, insomnia, |
| | system | migraines, depressions |

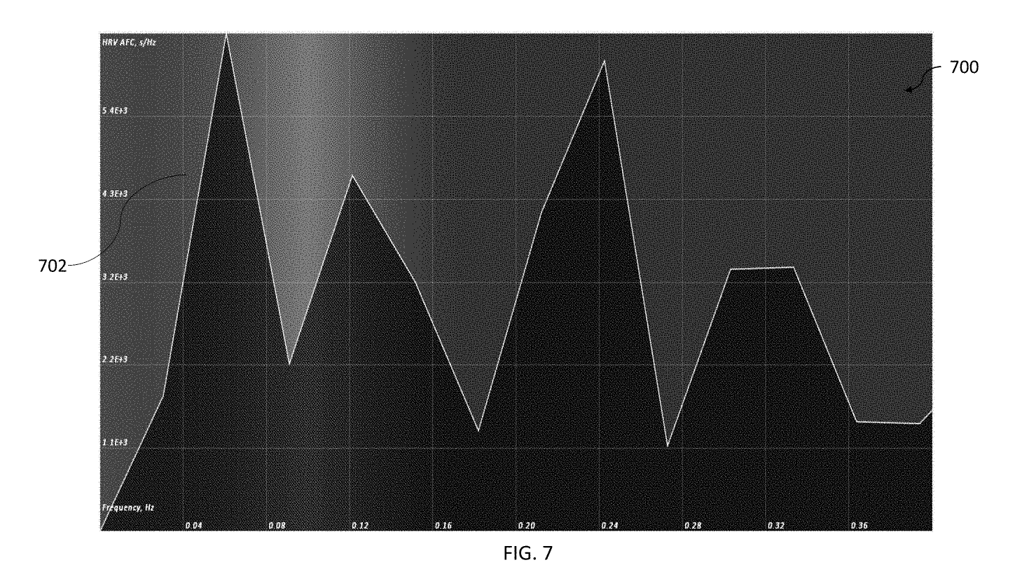

The frequency (wavelength), i.e., color, is determined based on the obtained data on the functional conditions or user's desire to achieve the required effect. For example, to calm or steady the user's nerves (indicators of the sympatric nervous system predominance, stress), the processor 208 executes program instructions to determine the properties of a color(s): frequency (wavelength), phase, intensity, and range of colors, which are calculated based on the obtained data from the database 207 based on the cardiointervalogram analysis. FIG. 7 is a screenshot 700 of a final (post-wave) signal 702 after processing the cardiointervalogram of a particular person, showing the four principal peaks, frequency and amplitude (power), corresponding to the principal waves ULF, VLF, LF and HF for an exemplary health condition for which preventive treatment and functional correction for this condition is implemented in accordance with an aspect of the invention.

Referring again to FIG. 3, in step 316 the processor 208 executes program instructions to automatically select the parameters for color rhythmic stimulation. For example, functional correction of a user's conditions may include overcoming fatigue, normalizing appetite, increasing physical activity, coping with jet-lag symptoms, and the like. In this regard, the red color is determined based on the pulse wavelength: (1) the time for brightness changing between an initial value of 0 to a maximum value on the scale of 250 c.u., which corresponds to the heart rhythm (nearly 0.8 seconds); (2) the duration of the maximum brightness luminance period (nearly 0.8 seconds); and (3) the time period for brightness changing from the maximum value of 250 c.u. on the scale to 0 (nearly 0.8 seconds). The green color is determined based on the breath wave time for brightness changing from an initial value of 0 to a maximum value on the scale (of 250 c.u.) corresponding to 198, which equates to 3.8 seconds, and the duration of maximum luminance is 3.8 seconds. The time period for the brightness to change from the maximum value of 198 to 0 is also 3.8 seconds. The yellow color is determined from the first-order waves, and the blue color is determined from the second order waves. The following table depicts the technical characteristics of the illustrative modulated color signal:

| | | | | Intensity | | |
|---|---|---|---|---|---|---|
| | Period of a | Period of | Period of | (transparency | | |
| | brightness | bright | signal | scale 250 | | |
| Emitter color | taking-up | luminance | extinction | c.u.) | Phase | Frequency |
| ULF red | 32.2 sec. | 32.2 sec. | 32.2 sec. | 45% | 10 | 0.031 Hz |
| VLF yellow | 15.4 sec. | 15.4 sec. | 15.4 sec. | 100% | 20 | 0.065 Hz |
| LF green | 8.3 sec. | 8.3 sec. | 8.3 sec. | 67% | 30 | 0.11 Hz |
| HF blue | 4.76 sec. | 4.76 sec. | 4.76 sec. | 92% | 40 | 0.21 Hz |

In step 318, the processor 208 executes program instructions to select the properties of acoustic waves (i.e., frequency, amplitude, time of exposure, etc.), for acoustic rhythmic stimulation. In an exemplary application, it has been established that an application of an acoustic signal of 77.7 Hz on the human body activates serotonin and dopamine receptors by modulating the slow waves obtained by cardiointervalography. For example, an acoustic signal of 77.7 Hz with a duration of 6 minutes is generated. A change in the signal amplitude (volume) will correspond to the wave characteristics of the second order waves equal to 0.021 Hz, which in turn corresponds to approximately a one-minute rhythm. The sound volume reaches a maximum value in 15 seconds, after which it decreases to a minimum value in 30 seconds, and then increasing again to a medium value in 15 seconds. While modulating the second-order waves, the first-order waves are modulated where 0.11 Hz corresponds to a nearly 10 second rhythm, the next modulation has approximately a 4 second rhythm, and the subsequent modulation has approximately a 1 second rhythm. Additional examples are described below.

In step 320, the processor 208 then stores these specified properties of the light and/or acoustic waves in the memory 206 to be applied to the consumable liquid based upon the determination of these properties by modulating the light and acoustic waves using the principal waves obtained via the cardiointervalogram.

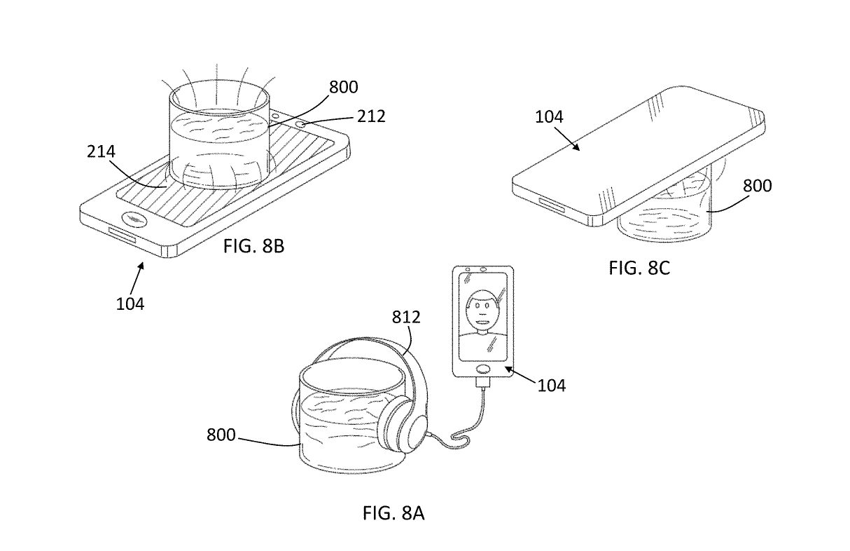

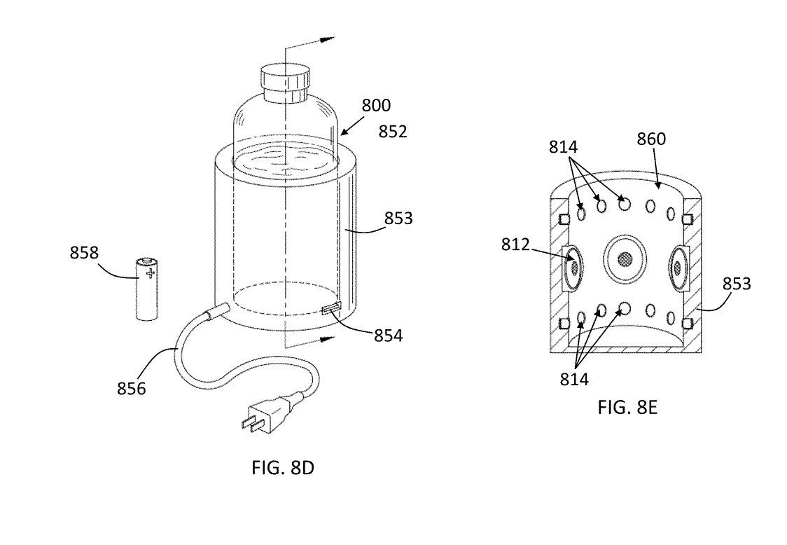

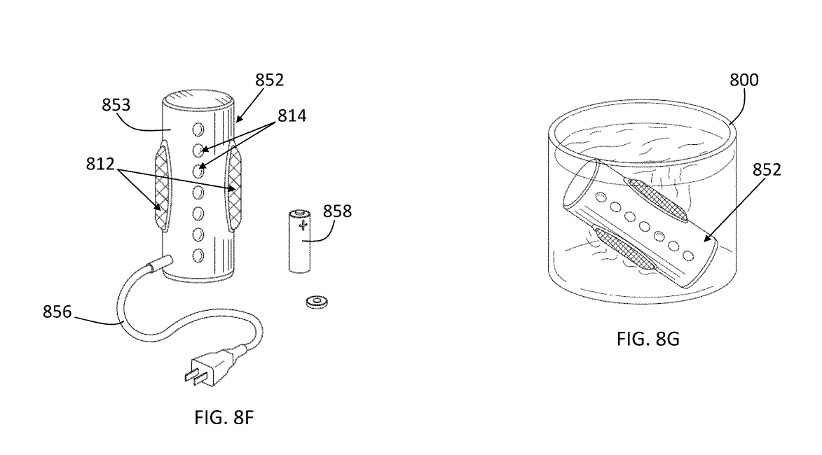

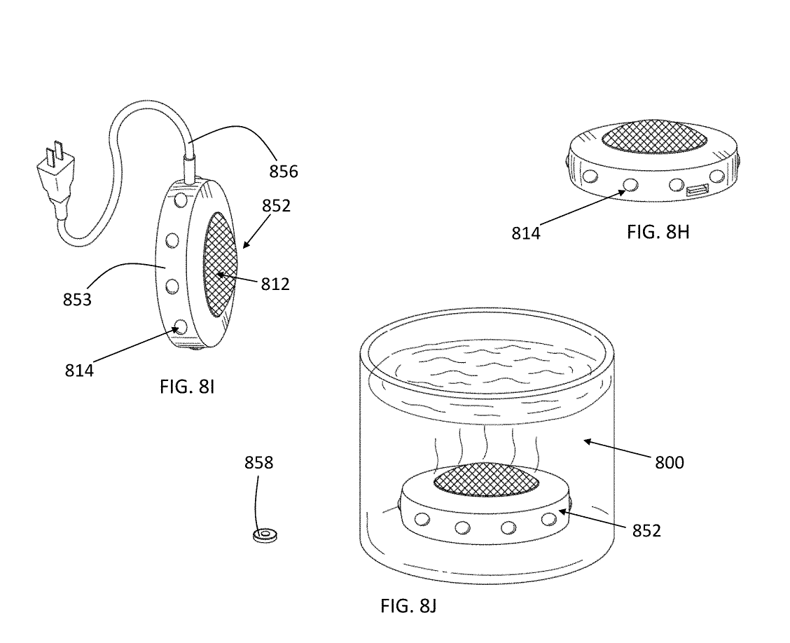

In step 322 and with reference to FIGS. 8A-8J, there are depicted a plurality of exemplary embodiments for exposing a consumable liquid, such as water 800, to an audio source 212 and/or light source 214 of the personal computing device 104, or a peripheral device communicating with the personal computing device over a wired or wireless short range communications protocol. The water is depicted within a generic transparent container having, for example, a volumetric capacity of 4-8 fl. oz.

In a first exemplary embodiment depicted in FIG. 8A, a transparent container 800 containing a consumable liquid is placed on a support surface, and a pair of headphones 812 are positioned around the periphery of the container against the outer surface 850 thereof. In this expedient, the headphones 812 are connected to the personal computing device 104 through a wired link for emitting sound waves having specified properties to modify the properties of the consumable liquid. Alternatively, the headphones may be coupled to the personal computing device 104 via a short-range communications protocol such as Wi-Fi or Bluetooth, as is well known in the art.

FIG. 8B is an illustration of another embodiment in accordance with an aspect of the present invention, in which the personal computing device 104 is placed on a support surface, and the transparent container 800 is disposed over the display 210 of the personal computing device. In this expedient, the display 210 is operable to project light waves having the specified properties against the underside of the transparent container. Alternatively, or in conjunction with light waves emitted the display 210, the light waves may be communicated from light source 214 and sound waves may be directed from audio source 212. Yet further, the audio source 214 may comprise a source that emits vibrations through the body of the personal computing device having the specified properties directly against the container 800.

With reference to FIG. 8C, there is depicted an embodiment that operates in principal similar to that shown in FIG. 8B, where the transparent container 800 is placed on the support surface, and the personal computing device 104 is positioned over the transparent container 800 with the display 210 face down against the top of the container.

Referring now to FIG. 8D, there is pictorial illustration of another embodiment in accordance with an aspect of the present invention, in which a peripheral device 852 configured to emit light waves and/or sound waves is constructed and arranged to receive and surround a transparent container 800. The peripheral device 852 is configured with a generally cylindrical housing and, in the exemplary embodiment, is shown having a Bluetooth port 854 for facilitating a wired connection to the personal computing device 104. Alternatively, the peripheral device 852 may be provided with capability to couple to the personal computing device over a short-range communications protocol such as Wi-Fi and/or Bluetooth. The peripheral device 852 may be powered via the USB connection, an external power supply 856, or battery(s) characterized generally by the reference numeral 858. FIG. 8E is a cutaway view of the peripheral device 852. In the illustrative embodiment, the peripheral device 852 is provided with an inner bore 860 for receiving the transparent container 800 (FIG. 8D), and a plurality of circumferentially disposed light sources 814 are positioned along the inner bore 860 to project light waves against the transparent container 800. Said another way, the bore 860 is shaped and/or sized to receive he container 800. In the expedient shown in FIG. 8E, the light sources 814 are arranged in two rows that are spaced apart and which completely extend around the circumference of the inner bore 860. Likewise, a plurality of audio sources 812 are positioned between the two rows of light sources 814 and extend around the circumference of the inner bore. It will be appreciated by those skilled in the art, that the configuration depicted in FIG. 8E is intended to be exemplary, and that various modifications to the arrangement of the audio sources 812 and light sources 814 may be made without departing from the scope of the invention. For example, the audio sources 812 may be positioned above and below the light sources 814, or the peripheral device may be singularly provided with an audio source 812 or a light source 814, in lieu of the combination shown and described. Similarly, the housing 853 could encompass other external configurations, with the cylindrical form being just one example.

FIG. 8F is an illustration of yet another embodiment of a peripheral device 852 in the shape of a “stick” for carrying out aspects of the present invention. In the example embodiment, the peripheral device 852 is constructed and arranged for placement into the consumable liquid contained within the container 800, as shown schematically in FIG. 8G. The peripheral device can be waterproofed techniques known to those skilled in the art, such as those utilized with waterproofing smartphones, tablets, external speakers and the like, and thus configured for operating while immersed within the consumable liquid. It will be appreciated that in this embodiment, the container 800 need not be transparent. The peripheral device 852 as shown, contains a generally cylindrical housing 853, and a plurality of spaced light sources 814 longitudinally disposed along the length of the housing 853. The light sources 814 may be provided at other locations (not shown) along the length of the housing 853, which are spaced apart at specified circumferential locations, i.e., 45 degrees (4 rows), 60 degrees (3 rows), 90 degrees (2 rows), and the like. In the sample embodiment, a pair of audio sources 812 are positioned on the housing 853 as shown. Again, it will be appreciated by those skilled in the art that the configuration and position of the audio sources 812 and light sources 814 on the housing 853 can be modified within the scope of the invention and the depicted embodiment is merely exemplary. As described above with respect to the embodiment shown in FIGS. 8D and 8E, the peripheral device shown in FIGS. 8F and 8G can similarly be provided with a USB port 854 to couple to the personal computing device 104 through a wired connection, and/or may further be provided with Wi-Fi and/or Bluetooth functionality to communicate with the personal computing device 104 through a wireless connection. Likewise, the peripheral device 853 may be powered via a USB connection to the personal computing device 104, an external power source 856 (110V, 220V), a battery 858, or the like.

FIGS. 8H through 8J illustrate yet another exemplary embodiment of a peripheral device 852 in accordance with an aspect of the present invention. In this embodiment, the peripheral device 852 is constructed and arranged as an immersible “tablet” having a generally cylindrical body 853. The peripheral device 852 is inserted into the consumable liquid within the container 800 as shown in FIG. 8J. In this embodiment, a plurality of light sources 814 are circumferentially and uniformly positioned around the periphery of the body 853, and an audio source 812 is centrally disposed on one side of the body 853. Here again, the positioning of the light sources 814 and audio sources 812 is intended to be merely exemplary. Those skilled in the art will appreciate that other arrangements may be incorporated within the scope of the present invention, such as multiple rows of light sources 814 and/or a plurality of audio sources 812 with each audio source 812 disposed on opposite sides (top and bottom) of the body 853. Alternatively, both the light sources 814 and audio source(s) 812 may be positioned circumferentially about the body 853 similar to the embodiment of FIGS. 8F and 8G, or the light sources 814 could be positioned on the top and bottom sides of the body 853, with the audio source(s) 812 circumferentially positioned about the body 853. Similar to the embodiments described above and illustrated in FIGS. 8D through 8G, the peripheral device 853 of FIGS. 8I through 8J may be provided with a USB port 854 to connect to the personal computing device 104. Alternatively or in conjunction therewith, the peripheral device 853 may additionally be provided with Wi-Fi and/or Bluetooth functionality to communicate with the personal computing device 104 through a wireless connection. Similar to the embodiments described above, the peripheral device 853 may be powered via a USB connection to the personal computing device 104, an external power source 856, a battery 858, or the like.

In step 324, the audio source 212 and/or light source 214 emits at least one of light and acoustic waves having the specified properties as determined from the slow waves of the cardiointervalogram to the consumable liquid so as to modify properties of the consumable liquid to preventively treat and functionally correct the particular human health condition identified by the cardiointervalogram as set forth above. The process ends at step 326. Using water as an exemplary consumable fluid, the primary fluid properties that are to be modified by this process are the following:
electronic water saturation (change of oxidation-reduction potential, pH factor, and electrical conductivity);
biocatalytic activity (content of oxygen ion radicals);
thermodynamic changes (dynamic viscosity, enthalpy and entropy);
water memory;
water memory density distribution by energetic levels.

Water subjected to such physical influence (light and acoustic waves) acquires new properties that have an effect on the occurring cooperative processes, including electron condensation and oxygen active forms, decomposition of such compounds and subsequent electron recondensation, and change in macrophysical properties and physical activity related to these processes.

This modified liquid, when consumed, has an impact on biological objects and thus can preventively treat and functionally correct human health conditions. The external physical and chemical factors impact is caused by absorption by a biological object of the corresponding factor energy, migration and transformation of such energy into biological reactions. One of ordinary skill in the art will appreciate that such transformations include the following physical and chemical changes and effects:
temperature effect (heat generation);
ion shifts;
generation of substance free forms;
electrical polarization;
bioelectric effect;
free-radical processes (generation of free radicals);
conformational changes; and
water state changes.

In accordance with aspects of the present invention, the properties of water are advantageously modified through synchronized application of electronic quantum and acoustic signals determined using the wave characteristics of human body activity identified by the cardiointervalogram. The process enables stimulation of oxygen active which forms generation of hydroxyl-radicals (OH*), super-oxide radicals (O2*), and peroxide radicals (OH2*). These are responsible for all oxidation-reduction processes occurring in protein-based and non-protein based structures, while concurrently activating the metal salt valent state.

Structurally, protons are distributed in water in the form of ion-crystalline associates located strictly perpendicular to interphase phases. In addition to donor-acceptor functions, these associates also function as superconductors for electromagnetic fields emitted by various external sources.

In addition to proton activity, the carriers of a non-compensated negative charge—OH-ion-hydroxyls—play a significant role in liquid media. Research has shown that the hydroxyls effect on water leads to an increase in heat capacity and a decrease in surface traction. But these phenomena are not strictly related to recovery of the structure of ion-crystalline associates formed by OH— and located strictly collinear and equidistant to interphase phases. Strengthening of H₃O ions structural organization under the effect of ion-crystalline associates of the OH— group leads to activation of proton movement in the H₃O composition when the oxidation process takes place.

This process underlies the mechanism of mediated quantum therapy, color therapy and luminotherapy, based on directed effect of small amounts of electromagnetic emissions on water for the purposes of human preventive treatment using the factors of electromagnetic impact similar to natural factors.

The efficacy of electromagnetic emissions on a liquid such as water in various applications is known. For example, the treatment of wastewater with electromagnetic emissions to reduce the pollutant level is disclosed in the publication “The study of Electromagnetic Waves on Industrial Wastewater”, M. Sirinivasa Rao, et al., Departments of Chemical and Electrical Engineering, NIT, Raipur (2013), the contents of which are incorporated by reference.

The subdivision of the electromagnetic spectrum comprising far infrared radiation (wavelengths=3-100 μm) has investigated for therapeutic effects on human health as described in “Far infrared radiation (FIR): its biological effects and medical applications,” Fatima Vatansever, et al., U.S. National Library of Medicine (2012), the contents of which are incorporated by reference. This publication describes the interaction between FIR and human cells, and the medical implications of such treatment.

The treatment of drinking water with ultraviolet (UV) emissions is disclosed in “Drinking water treatment with ultraviolet light for travelers—Evaluation of a mobile lightweight system,” Lisa F. Timmermann, et al., Travel Medicine and Infectious Disease, Vol. 13, Issue 6, pages 466-474 (2015), the contents of which are incorporated by reference. In this publication, there is described a device that applied UV radiation emissions to different types of bottles storing water containing a known number of microorganisms (Escherichia coli, Staphylococcus aureus, and the spore of Geobacillus stearothermophilus). After treatment, the survival rate of the organisms was examined.

The effects of magnetic and electromagnetic emissions on water are further described by Martin Chaplan at http)://www1.lsbu.ac.uk/water/magnetic_electric_effects.html, which describes how these fields affect the structure of water, specifically, due to the partial covalency of hydrogen bonds in the water.

Drinking water is one of the requirements for production of neurotransmitters and peptide hormones in human body that determine human energy balance and homeostasis, which depends primarily on epigenetic conditions (landscape), i.e. on the state of extracellular and intercellular biological substances and chemical elements containing water.

In accordance with an aspect of the present invention, the processor 208 executes program instructions to segregate the principle waves into the following:
pulse wave with cyclic rate of approximately 1 second;
breathing waves with a breathing cyclic rate of 3 to 5 seconds;
first-order waves or Traube-Hering waves with a cyclic rate of 10 to 12 seconds; and
second-order waves from 30 to between 70 to 90 seconds or Mayer waves.

The first-order waves in the period from 10 to 12 seconds (S1) reflect control of internal body regulation of intrasystemic processes. The second-order waves in the period from 30 seconds to several hours (S2) characterize systemic processes and interaction with a medium. The longer the control period, the higher the resolution.

These subordinate regulatory processes that proceed at a faster rate. Thus, for example, a transition to a condition of unsatisfactory adaptation of a body system is followed by, but not limited to, a drop off in the amplitude of breathing waves in the spectrum, and an increase in wave activity in the range over a period of approximately 30 seconds. In this regard, it will be appreciated that a frequency of up to approximately 5 seconds may be used to stimulate autonomous control loops, such as the cardiovascular system, under conditions of body disadaptation or correction of a stressful or overstressed state. On a metabolic level, undesirable body conditions such as disadaptation are followed by disturbance of oxidation and phosphorylation, which in turn leads to activation of free radical oxidation associated with ribosomes labialization and release of proteolytic enzymes from the ribosomes to cytoplasm and blood plasma with accompanying Ca++ emission.

The range of between 10 and 12 seconds, includes a nearly 10 second wave, which is understood to be responsible for control over the state of vascular tone via smooth muscle contraction of the vascular walls. Thus, the waves can be utilized as an indicator of sympathetic and parasympathetic balance, and as a reflection of all arterial pressure regulation system activity. Stimulation with a frequency of 0.1 Hz and below can assist in training of vascular smooth muscle wall contraction activity and reduction of peripheral vascular resistance, concurrently reducing stress on the heart and normalizing arterial pressure during periods of difficult intellectual or physical activity.

There will now be described several examples where a consumable liquid is modified using aspects of the present invention to preventively treat some exemplary conditions. In the first example, a tested person has a DSM-5 Somatoform or somatic disorder, which are related to increases in arterial pressure, e.g., 170/100. The tested person was positioned in front of the camera 200 at a maximum distance of 1 meter. The experiment was conducted at a temperature of 24 C, an atmospheric pressure of 1223 gPa, and humidity of 63%. From the images/video recorded by the camera 200, the processor 208 generates the cardiointervalogram, and processes the pulsogram with the obtained data with the four principal peaks ULF, VLF, LF and HF, as described above.

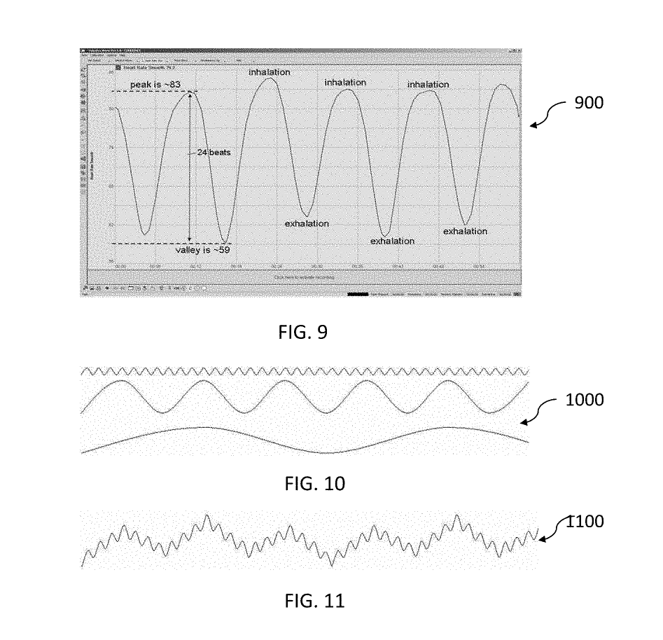

Referring to FIGS. 9 through 11, the stages of pulsogram processing are graphically depicted. FIG. 9 pictorially illustrates the pulsogram (HRV) 900 obtained for a test subject, FIG. 10 depicts the characteristics specifying the main parameters of an acoustic component (wave) 1000, and FIG. 11 shows post the slow wave parameters 1100 obtained from post transformation. In this example, a carrier frequency of 800 Hz is utilized for arterial pressure correction. The change in water properties was evaluated based upon changing the oxygen active forms parameters. A 150-ml water sample was employed. The acoustic waves applied to the water were generated by modulating the carrier frequency of 800 Hz by two channels for a period lasting up to 330 seconds using the amplitude of the slow waves (ULF, VLF and LF) from the cardiointervalogram. Next, the processor created a color frame using the corresponding color brightness take up from the slow waves, with a violet color having a period of 333 seconds, a green color having a period of 104 seconds, a yellow color having a period of 27 seconds, and a blue color having a period of 4.2 seconds. These acoustic and light waves were applied to the 150-ml water sample. The test subject was provided with the modified water and arterial pressure was measured in 5 minute intervals. Normal arterial pressure was observed in 15 to 20 minutes.

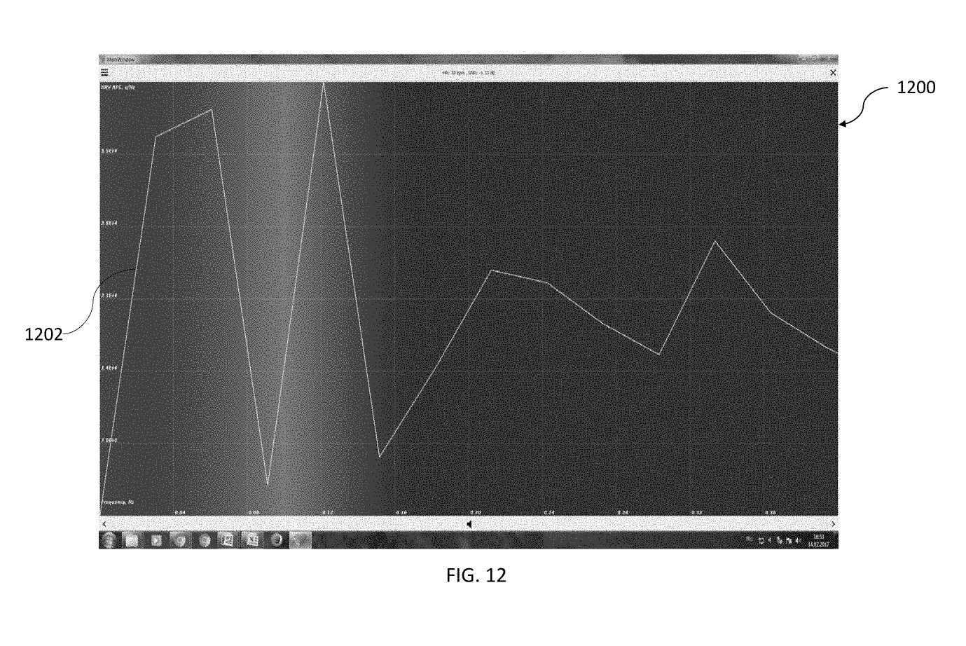

In the next example, the inventive process was applied to a test subject in a stressed condition. FIG. 12 depicts a screenshot 1200 of the pulsogram 1200 that reflects this state. The subject had a regulatory and adaptation status index on the second level, was unable to make correct decisions, and exhibited increased nervous excitation. An evaluation of the state of the stress was performed via electrocardiography and stress resistance was determined based on dynamics of heart and breathing parameters, range of synchronization, time period for development of heart and breathing synchronism at the minimum range border. The regulatory and adaptation status index (RASI) was calculated by the formula: RASI=SR/RpDmin.b. 100 (synchronization range/time period for synchronization development at the minimum range border), and regulatory and adaptation abilities of the body were determined on the basis of this index. The obtained data and estimated values were then processed using statistical methods of direct and indirect differences. Using these values, a water sample was modified using acoustic range frequencies close to 200 Hz, and color range of the blue-green-violet spectrum. The test subject consumed the modified water and the regulatory and adaptation status index shifted to the 5^{th} level, which indicates that the test subject was provided with working internal protection and better stress resilience.

In another example, a healthy person who desired to lose weight was tested. Using the obtained cardiointervalogram, the characteristic of the slow waves (ULF, VLF and LF) were used to generate acoustic waves by modulating a carrier frequency of 41 Hz by two channels over a period of 330 seconds. An orange-yellow-green spectrum was selected using the principals described above, and the specified acoustic and light waves were applied to a 150-ml water sample to modify the properties thereof. The test subject drank 150 ml of the activated water 3 times per day, 20 minutes prior to meals, for a period of 6 weeks. With no changes or limitations on food quality or quantity, the test subject lost weight by 8 kg.

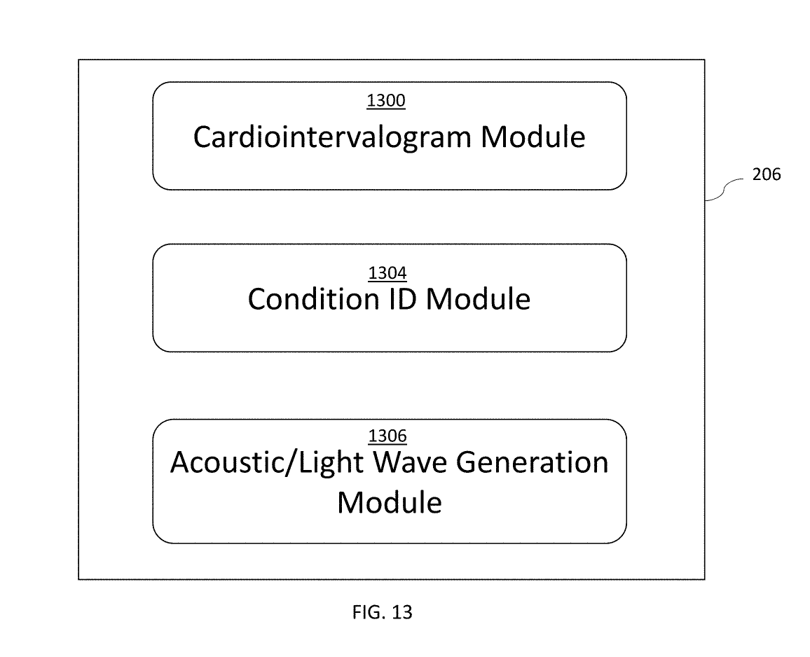

FIG. 13 schematically depicts a plurality of exemplary logical modules, where each logical module may include a set of computer instructions for executing various techniques and processes described herein. In the illustrative embodiment, there may be provided, for storage on, for example, the memory 206, a cardiointervalogram module 900 that processes acquired images and or video of a person and converts the data from those images into the person's cardiointervalogram, a condition identification module 902 for ascertaining a person's health condition for preventative treatment, and an acoustic and light generation module 904 for determining the specified properties for light and/or acoustic waves to be applied to the consumable liquid to preventively treat and functionally correct the particular human health condition, and directing the light source and/or audio source to emit light and/or acoustic waves having the determined specified properties to the consumable liquid to modify the characteristics thereof. As is understood in the art, these modules may be considered to be computer programming logic modules that are executed by a processing device, such as, the processing device 208. One of ordinary skill in the art will recognize that a computer or another machine with instructions to implement the functionality of one or more logical modules is not a “general purpose” computer. Instead, the machine is adapted to implement the functionality of a particular module. Moreover, the machine embodiment of the system physically transforms the electrons representing various parts of content and data representing user interaction with the content into different content or data representing determined resonance.

The operations described herein can be performed by an apparatus which may be specially constructed for the required purposes, or it may comprise a general-purpose computer selectively activated or reconfigured by a computer program stored in the computer. The apparatus may further include separate devices (modules) that can be immersed in a liquid or container, or a liquid may be surrounded by a special device configured to emit light and sound waves as described in FIGS. 8A through 8J above. A computer program may be stored in a computer readable storage medium, such as, but is not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on one computer, partly on the computer, as a stand-alone software package, partly on the first computer and partly on a remote computer or entirely on the remote computer or server. In the latter scenario, the remote computer may be connected to the first computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

A novel and efficient computer-implemented method and system has been disclosed that modifies the properties of a consumable liquid to preventively treat and/or functionally correct human health conditions based on a person's HRV. Having thus described the invention of the present application in detail and by reference to embodiments thereof, it will be apparent that modifications and variations are possible without departing from the scope of the invention defined in the appended claims as follows:

What is claimed is:

1. A computer-implemented method of modifying the properties of a consumable liquid to preventively treat or functionally correct human health conditions, the method comprising the steps of:
executing computer program instructions stored in a non-transitory memory, by at least one processor of an electronic device, to receive data representing a cardiointervalogram of a person;
identifying from the cardiointervalogram of the person, via the at least one processor of the electronic device, a particular human health condition of the person by comparing slow wave parameters of the cardiointervalogram to characteristics of corresponding human health conditions stored in a database;
determining, via the at least one processor of the electronic device, specified properties for at least one of light and acoustic waves to be applied to the consumable liquid to preventively treat or functionally correct the human health condition; and
from at least one of a light source and an audio source communicatively coupled to the processor of the electronic device, emitting at least one of light and acoustic waves having the specified properties to the consumable liquid to modify properties of the consumable liquid to preventively treat or functionally correct the particular human health condition.

2. The computer-implemented method in accordance with claim 1, further comprising:
capturing at least one visual image or video of a face of a person via a digital camera of the electronic device over a defined period of time; and
processing the captured at least one visual image or video, via a processor of the electronic device, to obtain the person's cardiointervalogram.

3. The computer-implemented method in accordance with claim 2, further comprising:
rendering on a display coupled to the at least one processor of the electronic device, the cardiointervalogram of the person in real time.

4. The computer-implemented method in accordance with claim 3, further comprising:
rendering on the display of the electronic device, a pulsogram of the slow wave parameters of the cardiointervalogram.

5. The computer-implemented method in accordance with claim 2, wherein:
the digital camera is operable to capture the at least one image or video of the person with a pixel resolution of at least 640×480 pixels.

6. The computer-implemented method in accordance with claim 2, further comprising:
prompting, by the electronic device, the person to be physically disposed in front of a camera lens of the digital camera with the face within a field of view of the camera lens during the defined period of time.

7. The computer-implemented method in accordance with claim 1, wherein:
the determining the specified properties for at least one of light and acoustic waves to be applied to the consumable liquid, comprises:
from the slow wave parameters of the cardiointervalogram and properties of light for preventively treating and functionally correcting the corresponding health conditions stored in the database, determining, via the at least one processor of the electronic device, selecting at least one color and properties of the color including frequency, phase and intensity, for emission to the consumable liquid.

8. The computer-implemented method in accordance with claim 1, wherein:
the determining the specified properties for at least one of light and acoustic waves to be applied to the consumable liquid, comprises;
from the slow wave parameters of the cardiointervalogram and properties of sound for preventively treating and functionally correcting the corresponding health conditions stored in the database, determining, via the at least one processor of the electronic device, selecting at least one sound and properties of the sound including frequency, phase and amplitude, for emission to the consumable liquid.

9. A computer-implemented in accordance with claim 1, wherein:
the consumable liquid is water and, by emitting the at least one of the light and acoustic waves having the specified properties, at least one of electronic water saturation, biocatalytic water activity, thermodynamic changes, water memory, and water memory density distribution by energetic levels of the consumable liquid is modified.

10. A system for modifying the properties of a consumable liquid to preventively treat or functionally correct human health conditions, the system comprising:
a database storing characteristics of human health conditions;
an electronic device communicatively coupled to the database and including at least one processor, a non-transitory memory, a display, an audio source, a light source, and a digital camera communicatively coupled to one another and having a set of computer program instructions stored in the non-transitory memory and executable by the at least one processor, the set of computer instructions including instructions for:
receiving data representing a cardiointervalogram of a person;
identifying from the cardiointervalogram of the person, a particular human health condition of the person by comparing slow wave parameters of the cardiointervalogram to characteristics of the corresponding human health conditions stored in the database;
determining specified properties for at least one of light and acoustic waves to be applied to the consumable liquid to preventively treat or functionally correct the particular human health condition; and
emitting at least one of light and acoustic waves having the specified properties, from at least one of the light source and the audio source to the consumable liquid to modify properties of the consumable liquid to preventively treat or functionally correct the particular human health condition.

11. The system in accordance with claim 10, further comprising:
computer program instructions for:
causing the digital camera to capture at least one visual image or video of a face of a person over a defined period of time; and
processing the captured at least one visual image or video to obtain the person's cardiointervalogram.

12. The system in accordance with claim 11, further comprising:
computer program instructions for rendering the cardiointervalogram of the person in real time on the display.

13. The system in accordance with claim 12, further comprising:
computer program instructions for rendering a pulsogram of the slow wave parameters of the cardiointervalogram on the display.

14. The system in accordance with claim 11, wherein:
the digital camera is operable to capture the at least one image or video of the person with a pixel resolution of at least 640×480 pixels.

15. The system in accordance with claim 11, further comprising:
computer program instructions for prompting the person to be physically disposed in front of a camera lens of the digital camera with the face within a field of view of the camera lens during the defined period of time.

16. The system in accordance with claim 10, further comprising:
computer program instructions for determining the specified properties for at least one of light and acoustic waves to be applied to the consumable liquid, by:
from the slow wave parameters of the cardiointervalogram and properties of light for preventively treating and functionally correcting the corresponding health conditions stored in the database, selecting at least one color and properties of the color including frequency, phase and intensity, for emission to the consumable liquid via the light source.

17. The system in accordance with claim 10, further comprising:
computer program instructions for the determining the specified properties for at least one of light and acoustic waves to be applied to the consumable liquid, by;
from the slow wave parameters of the cardiointervalogram and properties of sound for preventively treating and functionally correcting the corresponding health conditions stored in the database, selecting at least one sound and properties of the sound including frequency, phase and amplitude, for emission to the consumable liquid via the audio source.

18. The system in accordance with claim 16, wherein the light source is configured to emit light of variable properties including frequency, phase and intensity.

19. A system in accordance with claim 17, wherein the audio source is at least one of a speaker and headphones.

20. The system in accordance with claim 10, further comprising a peripheral device communicatively coupled to the electronic device by one of a wired and wireless connection, the peripheral device comprising at least one light source and at least one audio source for emitting the least one of the light and the acoustic waves having the specified properties to a container.
